(19) United States
(12) Patent Application Publication
(10) Pub. No.: US 2023/0039151 A1
(43) Pub. Date: Feb. 9, 2023

(54) Digital Healthcare Tracking and Coordination for Family and Friends

(71) Applicant: Wellscape LLC, Mount Laurel, NJ (US)

(72) Inventor: Nehal Trivedi, Mount Laurel, NJ (US)

(21) Appl. No.: 17/884,303

(22) Filed: Aug. 9, 2022

Related U.S. Application Data
(60) Provisional application No. 63/230,932, filed on Aug. 9, 2021.

(51) Int. Cl. G16H 10/65; G16H 15/00

(52) U.S. Cl. CPC G16H 10/65; G16H 15/00

(57) ABSTRACT

A Digital Family Care Tracking and Coordination (DFCTC) computing system associates a member with one or more care managers. The system stores member data on a cloud-based storage device. The one or more care managers are given access to the personal care data associated with the member via a mobile application. Member data includes medical information and relevant notes pertaining to a member's healthcare journey.

### PRIORITY

This application claims the benefit of U.S. Provisional 63/230,932, filed Aug. 9, 2021, which is hereby incorporated by reference as if submitted in its entirety.

### FIELD OF THE INVENTION

The present invention relates to systems and methods for facilitating real-time digital care tracking and management, and more particularly, to systems and methods for control of cloud-based systems including secure applications and data for the tracking and management of user data.

### BACKGROUND

In one's lifetime, a person may visit, beyond their primary care doctor for routine visits, many different types of specialists. Based on their medical condition, a patient may visit different medical centers (e.g., outpatient, hospital, rehab centers) and various specialists (e.g., OB/GYN, therapists, surgeons). Tracking and managing a patient's care is a monumental task that requires secure communication and storage.

### BRIEF SUMMARY OF THE INVENTION

A Digital Family Care Tracking and Coordination (DFCTC) computing system associates a member with one or more care managers. The system stores member data on a cloud-based storage device. The one or more care managers are given access to the personal care data associated with the member via a mobile application. Member data may include medical information and relevant notes pertaining to a member's healthcare journey.

### BRIEF DESCRIPTION OF THE DRAWINGS

This disclosure is illustrated by way of example and not by way of limitation in the accompanying figure(s). The figure(s) may, alone or in combination, illustrate one or more embodiments of the disclosure. Elements illustrated in the figure(s) are not necessarily drawn to scale. Reference labels may be repeated among the figures to indicate corresponding or analogous elements.

The detailed description makes reference to the accompanying figures in which:

FIG. 1 is a simplified functional block diagram of a computer system in accordance with the embodiments of the disclosure;

FIG. 2 illustrates an exemplary platform for facilitating at least one embodiment of the disclosure;

FIG. 3 illustrates an exemplary platform for profile management with at least one embodiment of the disclosure;

FIG. 4 illustrates an exemplary process in accordance with at least one embodiment of the disclosure;

FIG. 5 illustrates a simplified physical view diagram of a computing system in accordance with the embodiments of the disclosure;

FIG. 6 illustrates a simplified diagram of logical connections made by the disclosed architecture in accordance with an embodiment;

FIG. 7 illustrates another exemplary platform in accordance with at least one embodiment of the disclosure;

FIG. 8 illustrates yet another exemplary platform in accordance with at least one embodiment of the disclosure;

FIG. 9 illustrates an exemplary computing architecture in accordance with at least one embodiment of the disclosure; and

FIG. 10 illustrates another simplified physical view diagram of a computing system in accordance with the embodiments of the disclosure.

### DETAILED DESCRIPTION

The figures and descriptions provided herein may have been simplified to illustrate aspects that are relevant for a clear understanding of the herein described apparatuses, systems, and methods, while eliminating, for the purpose of clarity, other aspects that may be found in typical similar devices, systems, and methods. Those of ordinary skill may thus recognize that other elements and/or operations may be desirable and/or necessary to implement the devices, systems, and methods described herein. But because such elements and operations are known in the art, and because they do not facilitate a better understanding of the present disclosure, for the sake of brevity a discussion of such elements and operations may not be provided herein. However, the present disclosure is deemed to nevertheless include all such elements, variations, and modifications to the described aspects that would be known to those of ordinary skill in the art.

The disclosed system and methods described herein includes: a) a database that stores information about the care network including the primary care facilitator, helping co-facilitators and the members who are patients (whose care is being managed); b) a system to allow the facilitator, co-facilitator or member to capture, upload and store photos of insurance cards, medications, prescriptions, doctor's notes, test results and other medical record documents; c) programming logic to process the information to connect the member's underlying conditions with the relevant medication, providers (practices), treatment instructions, and visits to doctors, therapists, radiologists and other health services providers; d) front-end mobile and web experiences for facilitators and members to access, update, and manage this information; and e) a system to generate notifications or alerts for medications, treatment instructions, tasks and visits.

In some embodiments, the disclosed enables users, such as care receivers or the like, individually or in connection with other friends or family members to store, retrieve and track all relevant medical information, using consumer mobile devices such as a smartphone or tablet. Additionally, a graphical user interface, or web interface, may be provided on users' mobile devices. Interfaces provided may be device-agnostic and provide a similar user experience across multiple operating systems or device manufacturer. For example, a web interface design would make the same experience available over a browser on either an Apple® or Android® operating system.

In some embodiments, all data, notes, documents, and images regarding an individual's care is available via one application, putting the care receiver or their designated family members in control of their care. This is especially relevant for people who need help managing routine care, chronic conditions or transition care. Such care recipients would include elderly, those that are seriously ill, and young children, for example.

The disclosed embodiments take into account three factors: a) wide-spread, multi-generational adoption of smart-phones and tablets; b) capabilities of these devices; and c) ability of cloud-based backends to securely host data and documents as well as drive analytics and intelligence.

Our invention consists of creating an interactive, digital Family Care Coordination system that is accessible by consumers through mobile devices. The invention starts with creating a care profile that then allows creation of a personalized care plan. It then allows creation and maintenance of a digital care network connecting four stakeholders in-context of a care journey covering mental and physical health, including hospitalizations, rehab centers, skilled nursing and assisted living.

The four primary stakeholders, for example, may be: primary care facilitators (care managers), co-facilitators (helpers), members (care receivers), and Providers. The architecture creates a “care network” so that the primary facilitator (care manager) can invite other co-facilitators (helpers) to manage the logistics of care of one or more members (care receivers)

In some embodiments, facilitators are the primary users who sign-up and use the application to track the care of their friends and family members. Members are individuals who typically need help with their own medical care, and whose care is being facilitated and coordinated. Members may or may not be able to use the app and must totally rely on the Facilitators. Members include, but are not limited to, elderly parents, younger children, and spouses or siblings who need help. Members may also include family pets.

Providers are typically doctors and therapists working in medical practices. Providers include, but are not limited to, pharmacies, labs, and radiology practices. They can also include businesses who provide skilled and unskilled support services, home-care, assisted living, and companion services.

Each facilitator may have their own small network of members whose care they manage. A facilitator may also be a member. They may invite another individual as a co-facilitator for a particular member and the two will share the information of that specific member so that they can share the care related responsibilities.x

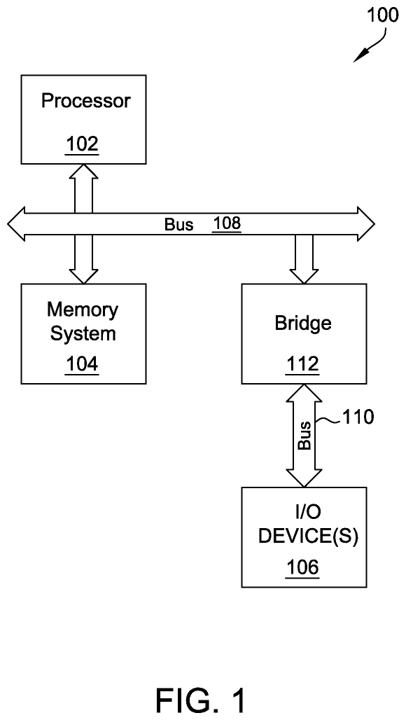

FIG. 1 is an example of a simplified functional block diagram of a computer system 100. The functional descriptions of the present invention can be implemented in hardware, software or some combination thereof.

As shown in FIG. 1, the computer system 100 includes a processor 102, a memory system 104 and one or more input/output (I/O) devices 106 in communication by a communication ‘fabric’. The communication fabric can be implemented in a variety of ways and may include one or more computer buses 108, 110 and/or bridge and/or router devices 112 as shown in FIG. 1. The I/O devices 106 can include network adapters and/or mass storage devices from which the computer system 100 can send and receive data for generating and transmitting advertisements with endorsements and associated news. The computer system 100 may be in communication with the Internet via the I/O devices 108.

Those of ordinary skill in the art will recognize that many modifications and variations of the present invention may be implemented without departing from the spirit or scope of the invention. Thus, it is intended that the present invention cover the modification and variations of this invention provided they come within the scope of the appended claims and their equivalents.

The various illustrative logics, logical blocks, modules, and engines, described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Further, the steps and/or actions of a method or algorithm described in connection with the aspects disclosed herein may be embodied directly in hardware, in a software module executed by a processor, or in a combination of the two. A software module may reside in RAM memory, flash memory, ROM memory, EPROM memory, EEPROM memory, registers, a hard disk, a removable disk, a CD-ROM, or any other form of storage medium known in the art. An exemplary storage medium may be coupled to the processor, such that the processor can read information from, and write information to, the storage medium. In the alternative, the storage medium may be integral to the processor. Further, in some aspects, the processor and the storage medium may reside in an ASIC. Additionally, the ASIC may reside in a user terminal. In the alternative, the processor and the storage medium may reside as discrete components in a user terminal. Additionally, in some aspects, the steps and/or actions of a method or algorithm may reside as one or any combination or set of instructions on a machine readable medium and/or computer readable medium.

The present embodiments may relate to, inter alia, systems and methods for facilitating real-time digital care tracking and management, and more particularly, to systems and methods for control of cloud-based systems including secure applications and data for the tracking and management of user data. In one exemplary embodiment, the process may be performed by one or more computing devices, such as a personal care computing device.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

The five characteristics are: on-demand self-service, broad network access, resource pooling, rapid elasticity, and measured service. On-demand self-service refers to the capability for a cloud consumer to unilaterally provision computing capabilities, such as server time and network storage, as needed automatically without requiring human interaction with the service's provider.

Broad network access refers to capabilities being available over a network and accessed through standard mechanisms that promote use by heterogeneous thin or thick client platforms (e.g., mobile phones, laptops, and PDAs).

Resource pooling refers to the provider's computing resources being pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity includes that capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service includes where cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

The three service models include: software as a service (SaaS), platform as a service (PaaS), and infrastructure as a service (IaaS).

Software as a Service (SaaS) includes where the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities with the possible exception of limited user-specific application configuration settings.

Platform as a Service (PaaS) includes where the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS) includes where the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

The four deployment models include: private cloud, community cloud, public cloud, and hybrid cloud.

Private cloud refers to a cloud infrastructure that is operated solely for an organization. It may be managed by the organization or a third party and may exist on-premises or off-premises.

A community cloud is a cloud infrastructure that is shared by several organizations and supports a specific community that has shared concerns (e.g., mission, security requirements, policy, and compliance considerations). It may be managed by the organizations or a third party and may exist on-premises or off-premises. A public cloud is a cloud infrastructure that is made available to the general public or a large industry group and is owned by an organization selling cloud services.

In a hybrid cloud the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

Turning now to an overview of technologies that are more specifically relevant to aspects of the disclosure, with the emergence and prevalence of remote cloud-based computing services, many companies and/or enterprises now use cloud-based computing services to run applications.

In at least one embodiment, a Digital Family Care Tracking and Coordination (DFCTC) computing system is provided. The DFCTC computing system allows individuals to use their mobile devices, such as smartphones, to facilitate and coordinate the care of their loved ones. This design enables users (care receivers), individually or in connection with other friends or family members to store, retrieve and track all relevant medical information, using consumer mobile devices such as Apple iPhones®, Android® Phones, iPads®, and other tablets, for example. The design would make the same experience available over a web browser on a personal computer (PC) or laptop.

All data, notes and documents regarding an individual's care may be available via single app, putting the care receiver or their designated family members in control of their care in a secure manner. This may be especially relevant for patients who need help managing routine care, care for chronic conditions, or transition care. Such care recipients may include, but certainly not limited to, the elderly, those that are seriously ill, the disabled, and young children.

The DFCTC computing system, in some embodiments provides the following technical advantages: a) wide-spread, multi-generational adoption of smart-phones and tablets; b) leveraging the capabilities of these devices; and c) ability of cloud-based backends to securely host data and documents as well as drive analytics and intelligence.

The DFCTC computing system may include systems and methods for digitally connecting multiple stakeholders in context of a care journey covering mental and physical health, including hospitalizations, rehab centers, skilled nursing and assisted living. The stakeholders, for example, may include care facilitators, members, and Service Providers. Facilitators may be users who sign-up and use the application to track the care of their friends and family members. Members may include patients for whom care is being facilitated and coordinated. Service Providers may include, for example, third parties who provide support services, home-care, medical or special transportation, elder care centers, or the like.

In some embodiments, each facilitator may have their own small network of members whose care they manage. A facilitator can invite another individual as a co-facilitator for a particular member and the two will share the information of that specific member so that they can share the care related responsibilities.

In some embodiments, five key components may be mapped to each other to drive an intelligent, personalized, guided experience for the duration of each member's care journey.

A members' personal care profile (PCP) may include, for example, insurance information, advanced directive information (DNR, DNI etc.) documents, reports (lab, radiology, hospitals), primary care and specialist doctors, therapists, and medication. Additionally or alternatively, the system may provide the tracking and coordination of certain events. The events may include, for example, visits, stays and medication prescriptions, and the management of chronic conditions and medical history details.

The DFCTC computing system may provide a mobile application that provides an easy way for a facilitator to create a complete profile and enter all relevant information for each member whose care they want to facilitate. This may be done with an understanding that the facilitator may not be a licensed medical professional. The app may rely on the facilitator for the accuracy, relevancy and timeliness of the information entered for each member.

The DFCTC computing system, in some embodiments, may provide value to care receivers and their family members. In some embodiments, the system may provide transparency and visibility into medical care history and upcoming visits and appointments across providers, health care systems, and insurance companies, for example. This may be realized by the system, for example, by facilitating appointments with primary care physicians, specialists, and therapists, ensuring that medications are taken as directed/needed, and providing referrals for nutritionists, transportation service and support groups.

The DFCTC computing system, in some embodiments, may provide value to medical service providers. Value may be realized, for example, by providing improved patient care where patients need help from family and friends to coordinate their care. Additionally, or alternatively, the DFCTC system may simplify referral intake process and reduce confusion when coordinating discharges and transitions, for example.

During an initiation process, a plurality of key assumptions may be made by the DFCTC system. The plurality of key assumptions may be made and disclosed to the individual signing up as a care facilitator. The facilitator must get consent from the member whose care is being tracked. The app, in at least one embodiment, may require the facilitator to confirm that they have the member's consent. For example, when the member has their own mobile phone number and email address, the app may send email and/or text notifications allowing the member to opt-out.

The accuracy and timeliness of care related information depends on the facilitator's diligence and attention to detail. State and Federal healthcare regulations will not prevent individuals who are not clinicians or licensed medical professions, from facilitating or coordinating care of their family members.

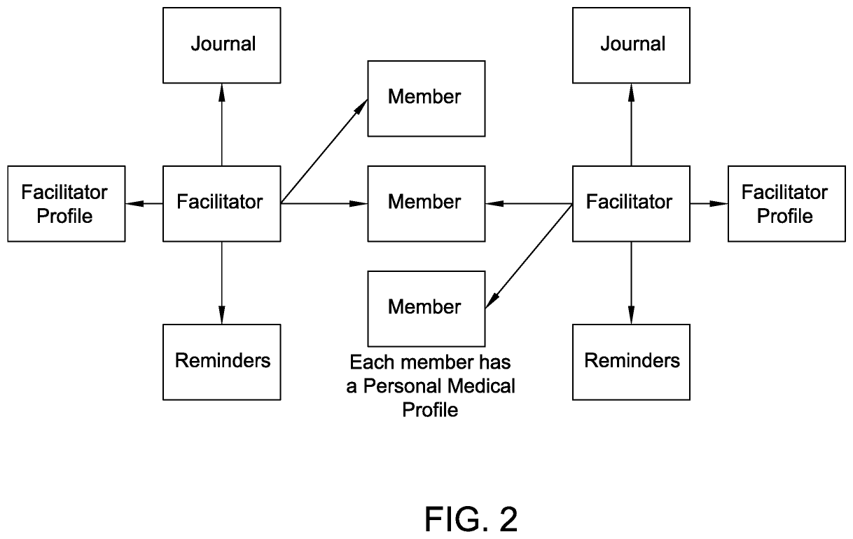

FIG. 2 illustrates a logical view implementation of the DFCTC computing system described herein. A facilitator may have access to multiple resources, such as a journal, a profile, and a set of reminders. In some embodiments, a facilitator may be logically connected to one or more members within the system. Further, one or more members may be logically connected to one or more facilitators.

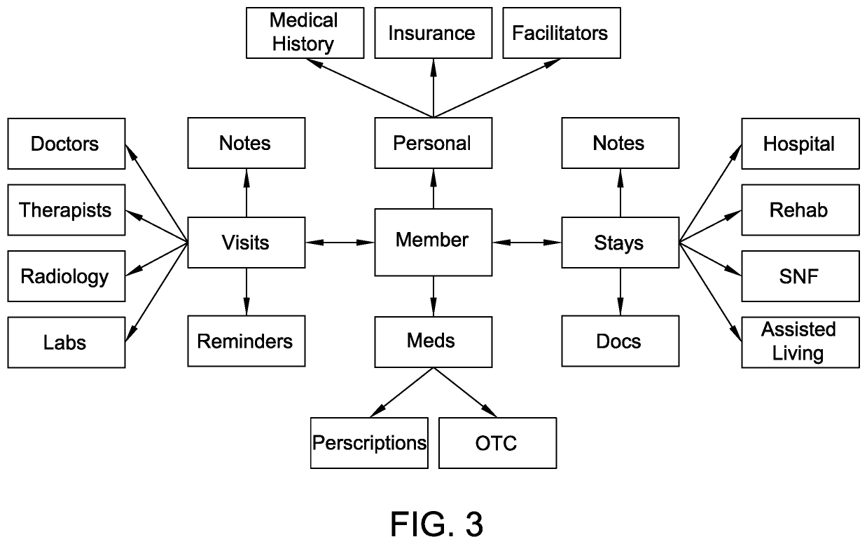

FIG. 3 illustrates a care journey a patient, or member, may undertake. The care journey may include multiple categories having their own sets of subcategories. For example, categories may include Personal Information (e.g., medical history, insurance, facilitators), Stays (e.g., hospital, rehab, notes, etc), Medications (e.g., Rx, OTC), and Visits (e.g., doctors, therapists, reminders, etc.). In some embodiments, information pertaining to a patient's care journey may be maintained on a cloud-based storage device.

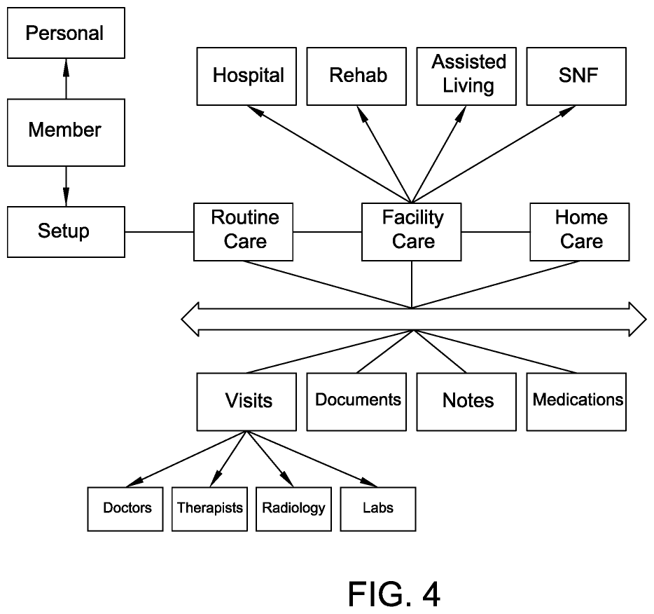

FIG. 4 illustrates a personal care profile (PCP) that may be maintained by the DFCTC system. As shown in FIG. 4, a profile may organize and categorize a patient's care journey, as described above in FIG. 3. On a base level, a patient may be setup to be provided with different types of care, such as routine, facility, and/or home, for example. In one embodiment, for example, facility care may include the management of sub-categories services, such as hospital, rehab, assisted living, or the like. In another embodiment, routine or home care may include sub-categories including, but not limited to, visits, documents, notes, and medications. Even further, visits may include another set of sub-categories including, but not limited to, doctors, therapists, and/or radiologists, for example. For each visit, data may be tracked. Tracked data may include, but is not limited to, appointment date and time, referral data (i.e. name of the doctor/therapist, link if the doctor/therapist profile already exists), reason: text field (80 characters), Diagnosis Code: alphanumeric field, and transportation (i.e. name of transportation company, ability to add it as a provider). Additional data may include any notes that were provided to the patient and follow up visit information. In some embodiments, the ability to add a document/image upload may be provided, such as an X-Ray image. For each doctor, therapist, radiology center, or lab test, there may be an opportunity to add notes or upload documents. The system may enable and facilitate back-end integration with EHR/EMR systems used by medical practices and hospitals. For each medication, especially prescription, there may be an opportunity to store key data points like dosage, quantity, frequency, number of refills, and any restrictions. Further, the system may allow a patient or caregiver to take a photo of the prescription label, if appropriate.

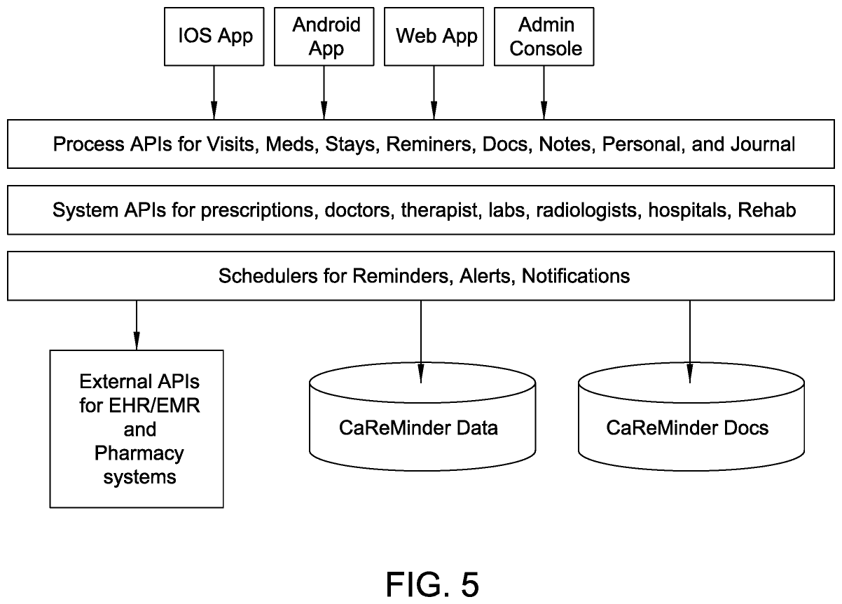

FIG. 5 illustrates an exemplary physical view of a DFCTC computing system described herein. For example, one or more APIs may be provided to facilitate an interface between care providers and their respective patients. For example, a process API may be provided to interface with a user to provide access to care data including, but not limited to, external third party devices, confidential data, and medical documents, for example.

Example journeys:

Routine Care Facilitation: checkups, primary care physician visits, vaccinations, ER visits, outpatient rehab, labs, radiology visits.

Chronic Care Facilitation: COPD, Heart Disease, Diabetes, Arthritis, Stroke,

Asthma, Alzheimers.

Transitional Care Facilitation: entering and leaving hospitals, inpatient rehab centers.

The present invention may be focused on average individuals, allowing then to directly coordinate, track and schedule activities and visits related to healthcare or wellness for themselves or others in their network, across medical practices, labs, pharmacies, healthcare networks and hospital systems. Healthcare scope would cover physical health, mental health, dental, and vision.

Similarly, patients may get prescriptions filled at different pharmacies or have their family members of friends help them get their medications. The present invention may provide systems and methods enabling an individual to manage real-time tracking, coordination and facilitation of activities related to healthcare, wellness and medical care, for themselves or their family members or friends.

In an exemplary embodiment, the primary user is the facilitator. The facilitator may access the system using a mobile device, such as an Apple® or Android® smartphone, for example. The mobile device, in some embodiments, may be a current generation phone that supports connectivity via wi-fi, 4G LTE cellular network, a 5G cellular network, or the like.

In an exemplary embodiment, a user may initiate facilitation by downloading an application from an app store, such as the Apple® App Store or the Google Play® store, for example. Next, the user would register with the system using personally identifiable data unique to them including, but not limited to, their email address and/or cell phone number. Additional identification data may be provided, such as home address, driver's license identification number, birthdate, confirmation number, health insurance data, etc.

After registration, the user may be provided with four choices:

1) Enrolling themselves

2) Enroll a new member

3) Link to an existing Member

4) Skip and go to the “home page”

Enrollment & Setting Up Member

Enrollment with the system may involve setting up the care profile to enable the facilitator to use the app to coordinate healthcare activities for the member or for themselves. In some embodiments, operating the app contemplates capturing basic information relevant to the member's care including, but not limited to, blood type, smoker status, last medical checkup, last dental checkup, last screening test such as colonoscopy or mammograms. This information can then be used to generate reminder notifications to the member and/or facilitator.

Further, operating the app further contemplates capturing and storing insurance information including, but not limited to, medical, dental, prescription and supplemental insurance. Capturing insurance information may be performed using a camera of the user's mobile device. For example, the camera may be used to take photos of an insurance card or other similar health insurance documents, such as proof of insurance documentation. The user's device may then upload photos or documents captured by the camera to the app.

Additionally, or alternatively, continued enrollment contemplates capturing and storing care related documents for the member. Care related documents may include, but are not limited to, Healthcare Power of Attorney, Advanced Directives, and DNI/DNR/DNH forms. In some embodiments, the app provides for providing information and reminders to facilitators and members about the importance of these care documents.

The enrollment process further contemplates capturing and storing member specific medical records, such as photographs or data files (e.g., PDFs). Medical records may include, but are not limited to, discharge instructions, provider's notes, lab results, or radiology reports. The system would allow a user to take a photo or upload an existing document image. The image or document may be stored in cloud file storage (e.g. AWS S3) and one or more tags may be stored in the cloud data storage associated with the member record.

Managing care by a user, or facilitator, may include a multitude of key components. There are three key components which may be mapped to each other to drive an intelligent, secured, personalized, guided experience for the duration of each member's care journey. A care profile typically covers, but is not limited to, contact information (email, phone etc.), insurance information, care related documents, reports (lab, radiology, hospitals), and visits.

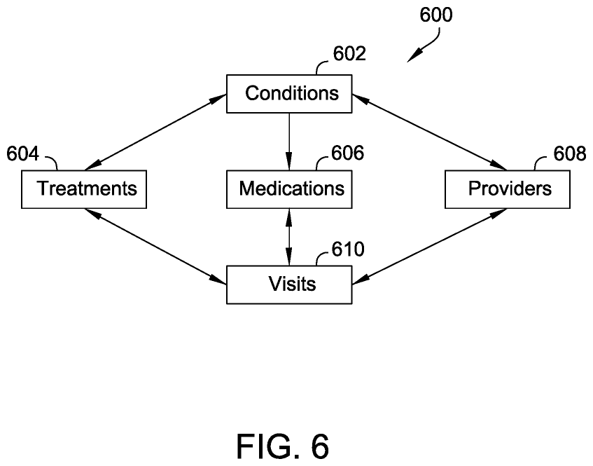

As depicted in FIG. 6, a care square 600 may be provided for each member of the system. Care square 600 connects underlying medical conditions 602 with medications 606, providers 608, treatments 604 (including activities of daily living) and visits 610 (e.g., provider visits).

For each member, or patient, the system connects what is being treated including medical conditions 602 (along with family history of the disease), who is doing the treatment 608 (e.g., doctor, therapist, dentist, optometrist), how the condition is being treated 604 (e.g., medications and treatments), and when is it being treated 610.

In this exemplary embodiment, rules may be added with respect to specific conditions and treatments that will help the facilitators create an interactive care plan for their family members. Additionally, or alternatively, meaningful analytics may be produced that when shared with the provider can impact patient satisfaction and understanding of the provider's instructions including follow-ups and referrals.

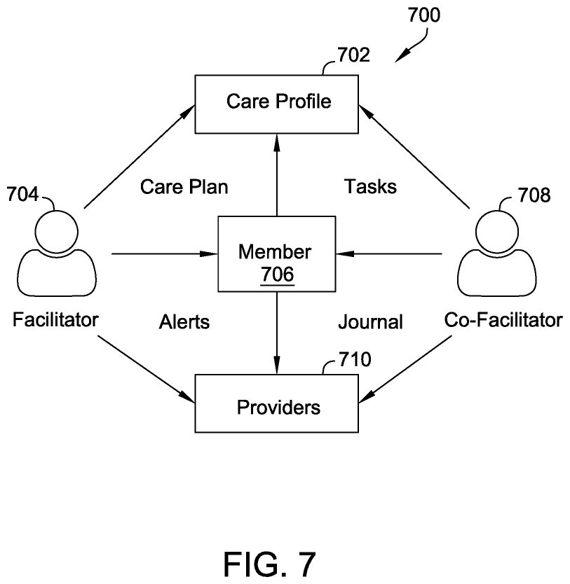

As depicted in FIG. 7, a care network 700 may be provided so facilitators 704 and co-facilitators 708 connect with members 706, share tasks via ToDos, share Journal for observations and get alerts for daily medication and treatment schedules. Daily medication or treatment schedules may be a part of a care profile 702 associated with a member 706 and shared with a provider 710.

In some embodiments, these three components may be backed up by a system of alerts and reminders (Care Alerts) that are implemented through push notifications and in-app notifications, for example. In some embodiments, notifications may be transmitting using email and text messages to deliver notifications related to these alerts. In one non-limiting example, a reminder may be triggered based on a medication schedule causing a notification to be transmitted to not just a member, but a facilitator as well.

In an exemplary embodiment, the mobile app may provide an easy way for a family member acting as the primary care facilitator for a user to create and complete a care profile 702 for a user. Via an interface on their mobile device, the primary care facilitator 704 may enter all relevant information for each member whose care they want to facilitate. This is done with an understanding that the facilitator has the consent of the member and may not be a licensed medical professional.

The mobile application would rely on the facilitator 704 for the accuracy, relevancy and timeliness of the information entered for each member's care profile 702. The system is intended to make it easier to shift the power of coordinating care to the family.

In the exemplary care network, each user may set up their own private care network. A private care network connects care facilitators 704 (primary care coordinator), co-facilitators 708 (helpers), members 706 (patients or care receivers) and providers 710 (3rd parties).

A care facilitator 704 may add members 706 (whose care you are helping with), co-facilitators 708 (who are helping you with the care of one or more members), and providers 710 (3rd parties who are providing healthcare, medical, or wellness services).

In some embodiments, the member 706 may, but does not need to, login and use the mobile application. Further, each member 706 may be provided with their own login credentials. Some members may be able to login and use the mobile application while others may not be able to. Additionally, members cannot invite other members but they can invite other co-facilitators or providers for their care.

A care profile 702 for each member may include, but is not limited to contact information, screening questions (life style, wellness, or social determinants), health care directives (POA, Advanced Directive etc.), and medical records (discharge instructions, radiology reports, lab reports etc.)

Additionally, a care profile 702 makes all relevant information available for sharing in the case of an emergency. It may also provide the foundation for tagging and searching for documents that can be shared with providers if approved by the member.

In some embodiments, an insurance information storage may be provided to store information and photos of insurance cards. This storage includes, but is not limited to, medical, pharmacy, dental, vision, and any supplemental insurances including, hospitalization and long term care.

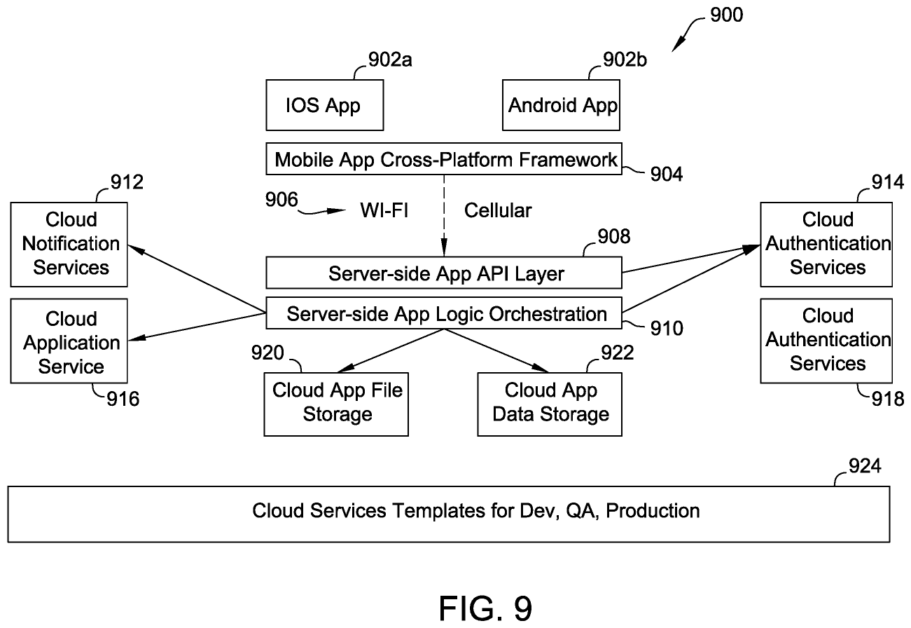

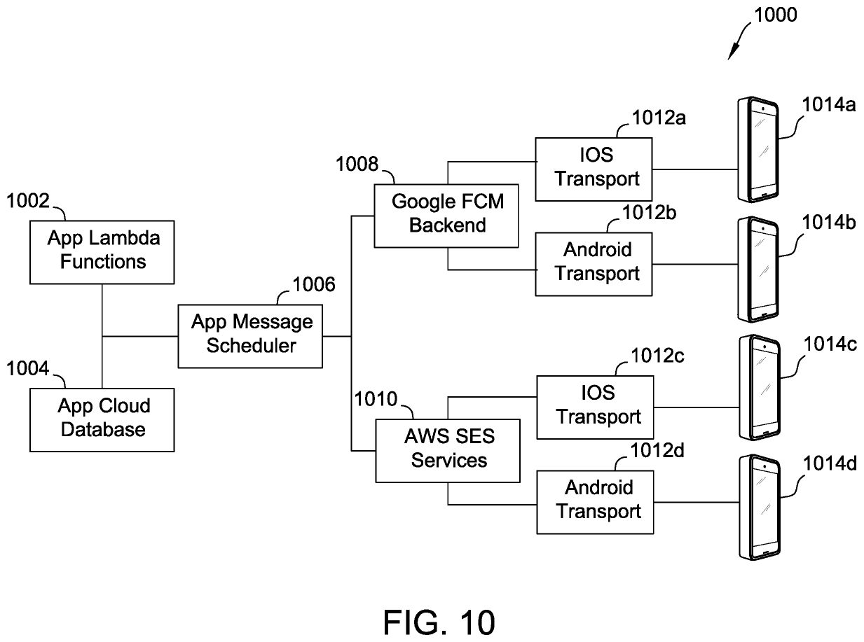

As depicted by FIGS. 9 and 10, in an exemplary embodiment, alerts, or digital nudging, may be provided. An active system 900 including a mobile app cross platform framework 904 may be provided. Framework 904 includes, but is not limited to, support for an iOS app 902a and an Android app 904a, for example. The system may be provided using alerts (or reminders) through push notifications and in-app notifications. For physical implementation, cloud-based messaging service, such as AWS SNS, AWS SES or Firebase Messaging Service, may be used.

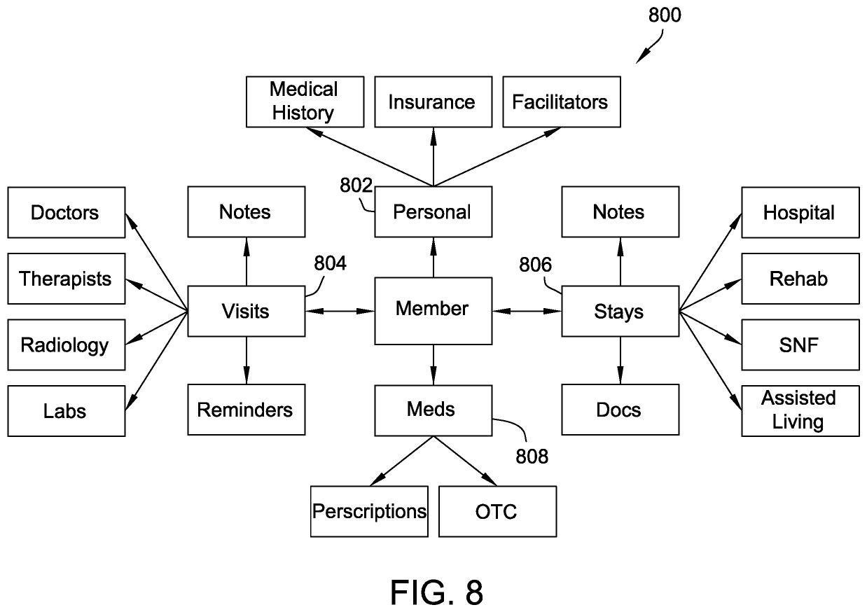

Alerts (or reminders) can be set up for medications, prescription refills, treatment instructions, visits, and tasks. The application contemplates a rule-based, profile-driven, scheduling engine to generate the alerts. Alerts may be sent to members as well as their facilitators and co-facilitators. FIG. 8 illustrates 800 logical connections between: a member's personal 802 data including, but not limited to, medical history, insurance, and medical providers; a member's stays 806; a member's visits; and a member's medications 808.

The system contemplates responsive alerts meaning that members or facilitators are allowed to respond to the alert notification to indicate whether the target of the alert is addressed and resolved. This allows generation of adherence reports that, with member consent, can be shared with providers treating the relevant condition.

The physical architecture described here is for the purposes of illustrating how the design can be implemented using cloud services. Current implementation is for Apple (iOS) and Google (Android) mobile devices with Amazon Web Services (AWS) server side backend. For the purposes of this utility and design, AWS could be replaced by any similar cloud services backend e.g. Google or Microsoft. For example, Google Firebase may be used interchangeably with AWS Simple Notification Service as the cloud notification services.

The presentation layer of the mobile app may be built using a cross-platform framework 904 and use wireless networking or cellular network technology 906. Exemplary technology may be React Native. Any other similar technology would serve the same purpose of rendering the mobile app screens driven by server-side business logic and data.

Server Side Layer 908 may be connected to the framework 904 via network 906. In some embodiments, all business logic and data may be accessed through secure API end-points. Custom business logic may be implemented on the app/client side using React Native, for example, and on the server side using AWS Lambda functions that talk to the underlying cloud app file storage 920 (Amazon S3) or cloud data storage 922 (Amazon DynamoDB) using server side app logic orchestration 910. Server side app 910 may be logically connected to one or more cloud services, such as cloud notification services 912, cloud authentication services 914 and 918, and cloud analytic services 916.

All server side functions and services may be configured and provisioned using serverless templates. Specific implementation uses AWS CloudFormation templates 924. For example, Serverless Application Model templates may be used.

FIG. 10 depicts a notifications layer 1000 that uses a systems of alerts and reminders for medications, treatments, tasks and visits. These alerts and reminders are implemented through push notifications and in-app notifications initiated by app lambda functions 1002 and app cloud database 1004. Both are specific to a user. Push notifications may originate from app message schedule 1006 and are specific to a device on which the app was downloaded and registered (e.g., Google® 1008 or AWS 1010). Alerts may be transported via an OS transport 1012a-1012-d and received on a device 1014a-1014d. They can be seen without opening the app. In App notifications are specific to the user and can be seen on any device the user is logged into and may also be seen inside the app. Both represent different forms of nudging.

It is appreciated that exemplary computing system 100 is merely illustrative of a computing environment in which the herein described systems and methods may operate, and thus does not limit the implementation of the herein described systems and methods in computing environments having differing components and configurations. That is, the inventive concepts described herein may be implemented in various computing environments using various components and configurations.

Those of skill in the art will appreciate that the herein described apparatuses, engines, devices, systems and methods are susceptible to various modifications and alternative constructions. There is no intention to limit the scope of the invention to the specific constructions described herein. Rather, the herein described systems and methods are intended to cover all modifications, alternative constructions, and equivalents falling within the scope and spirit of the disclosure, any appended claims and any equivalents thereto.

In the foregoing detailed description, it may be that various features are grouped together in individual embodiments for the purpose of brevity in the disclosure. This method of disclosure is not to be interpreted as reflecting an intention that any subsequently claimed embodiments require more features than are expressly recited.

Further, the descriptions of the disclosure are provided to enable any person skilled in the art to make or use the disclosed embodiments. Various modifications to the disclosure will be readily apparent to those skilled in the art, and the generic principles defined herein may be applied to other variations without departing from the spirit or scope of the disclosure. Thus, the disclosure is not intended to be limited to the examples and designs described herein, but rather is to be accorded the widest scope consistent with the principles and novel features disclosed herein.

1. A Digital Family Care Tracking and Coordination (DFCTC) computing system, with at least one computing device having a processor communicatively-coupled to a non-transitory computer readable medium having instructions stored thereon, wherein the instructions, when executed by the processor, are configured to implement:
associating a member with one or more care managers,
storing, on a storage device, personal care data associated with the member, and
providing, via a mobile application, the one or more care managers with access to the personal care data associated with the member.

2. The DFCTC computing system of claim 1, wherein the instructions are further configured to implement:
generating an alert based on the personal care data associated with the member.

3. The DFCTC computing system of claim 2, wherein the instructions are further configured to implement:
transmitting the alert to the one or more care managers associated with the member.

4. The DFCTC computing system of claim 3, wherein the instructions are further configured to implement:
generating an adherence report based on a response message received from at least one of the one or more care managers.

5. The DFCTC computing system of claim 1, wherein the personal care data associated with the members includes one or more of contact information, screening questions, health care directives, and medical records.

6. The DFCTC computing system of claim 5, wherein the personal care data is supplied by one other than the member.

7. The DFCTC computing system of claim 1, wherein the storage device is a cloud-based storage device.

8. A method for providing the facilitation of care across medical practices, healthcare networks and hospital systems, the method comprising:
associating a member with one or more care managers,
storing, on a storage device, personal care data associated with the member, and
providing, via a mobile application, the one or more care managers with access to the personal care data associated with the member.

9. The method of claim 8, further comprising:
generating an alert based on the personal care data associated with the member.

10. The method of claim 9, further comprising:
transmitting the alert to the one or more care managers associated with the member.

11. The method of claim 10, further comprising:
generating an adherence report based on a response message received from at least one of the one or more care managers.

12. The method of claim 8, wherein the personal care data associated with the members includes one or more of contact information, screening questions, health care directives, and medical records.

13. The method of claim 5, wherein the personal care data is supplied by one other than the member.

14. The method of claim 1, wherein the storage device is a cloud-based storage device.

15. A non-transitory computer readable medium comprising instructions that when executed by a processor implement:
associating a member with one or more care managers,
storing, on a storage device, personal care data associated with the member, and
providing, via a mobile application, the one or more care managers with access to the personal care data associated with the member.

16. The non-transitory computer readable medium of claim 15, wherein the instructions are further configured to implement:
generating an alert based on the personal care data associated with the member.

17. The non-transitory computer readable medium of claim 16, wherein the instructions are further configured to implement:
transmitting the alert to the one or more care managers associated with the member.

18. The non-transitory computer readable medium of claim 17, wherein the instructions are further configured to implement:
generating an adherence report based on a response message received from at least one of the one or more care managers.

19. The non-transitory computer readable medium of claim 15, wherein the personal care data associated with the members includes one or more of contact information, screening questions, health care directives, and medical records.

20. The non-transitory computer readable medium of claim 19, wherein the personal care data is supplied by one other than the member.

21. The DFCTC computing system of claim 1, wherein the storage device is a cloud-based storage device.
